(19) United States
(12) Patent Application Publication
(10) Pub. No.: US 2019/0037787 A1
(43) Pub. Date: Feb. 7, 2019

(54) Smart Moisture Meter

(71) Applicant: Lalit Kumar, San Jose, CA (US)

(72) Inventor: Lalit Kumar, San Jose, CA (US)

(21) Appl. No.: 16/047,120

(22) Filed: Jul. 27, 2018

Related U.S. Application Data
(60) Provisional application No. 62/540,836, filed on Aug. 3, 2017.

(51) Int. Cl. A01G 25/16; A01G 25/06; G01N 33/24; G01K 3/00

(52) U.S. Cl. CPC A01G 25/167; A01G 25/06; H04B 1/02; G01K 3/005; G01N 33/246

(57) ABSTRACT

A moisture meter having a housing with a stake disposed at a lower end thereof. A moisture sensor and a temperature sensor disposed on the stake. The moisture meter includes a valve wherein a water source is removably secured thereto. The stake includes a channel between the valve and a tip of the stake, allowing liquid to flow therethrough. Alternatively, the valve can be mounted on a wall or adjacent to a water line, wherein the stake can be connected to valve by a water hose. A wireless transmitter is disposed within the sensor housing and can transmit the moisture and temperature readings from the moisture and temperature sensors to an external source. A wireless receiver is disposed within the valve housing in order to actuate the valve, allowing water to flow through the stake and into the ground, should the moisture and temperature readings reach a pre-set level.

### CROSS REFERENCE TO RELATED APPLICATIONS

This application claims the benefit of U.S. Provisional Application No. 62/540,836 filed on Aug. 3, 2017. The above identified patent application is herein incorporated by reference in its entirety to provide continuity of disclosure.

### BACKGROUND OF THE INVENTION

The present invention relates to a moisture meter. Many people water their plants; however, it can be difficult to determine moisture content of the soil on a daily basis. This leads to plants getting watered when the moisture content is already sufficient, leading to overwatering, which can cause damage to the plants. Overwatering plants results in a considerable time investment for the user and wastes water. Further, water is a limited resource in certain areas, as well as during a drought, and in these situations, overwatering plants potentially causing expense to the user, or possible fines. Therefore, a device capable of monitoring the moisture content of soil and notifying a user when it reaches a pre-set level is needed.

Devices have been disclosed that relate to moisture meters. These include devices that have been patented and published in patent publications. These devices generally relate to a meter that measures the moisture level of soil. However, these devices fail to disclose a moisture meter with a wireless transceiver such as a Bluetooth, Wi-Fi or Cell Phone using Internet of Things (IoT) devices.

### SUMMARY OF THE INVENTION

In view of the foregoing disadvantages inherent in the known art it is submitted that the present invention substantially diverges in design elements from the prior art and consequently it is clear that there is a need in the art for an improvement to existing moisture meters. In this regard the instant invention substantially fulfills these needs.

It is therefore an object of the present invention to provide a new and improved moisture meter that has all of the advantages of the prior art and none of the disadvantages.

It is another object of the present invention to provide a moisture meter comprising a housing with a stake disposed at a lower side thereof; a moisture and a temperature sensor disposed on the stake.

Another object of present invention is to provide a moisture meter with a valve disposed on the housing wherein a water source can be removably secured to the valve; a wireless transmitter disposed within the housing and a wireless receiver disposed with the valve housing.

Other objects, features, and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the moisture meter.

FIG. 2 shows a perspective view of an embodiment of the moisture meter being inserted into soil.

FIG. 3 shows a close-up view of the temperature and moisture sensor of the moisture meter.

FIG. 4 shows a schematic diagram of the moisture meter.

### DETAILED DESCRIPTION OF THE INVENTION

Reference is made herein to the attached drawings. Like reference numerals are used throughout the drawings to depict like or similar elements of the moisture sensor. The figures are intended for representative purposes only and should not be considered to be limiting in any respect.

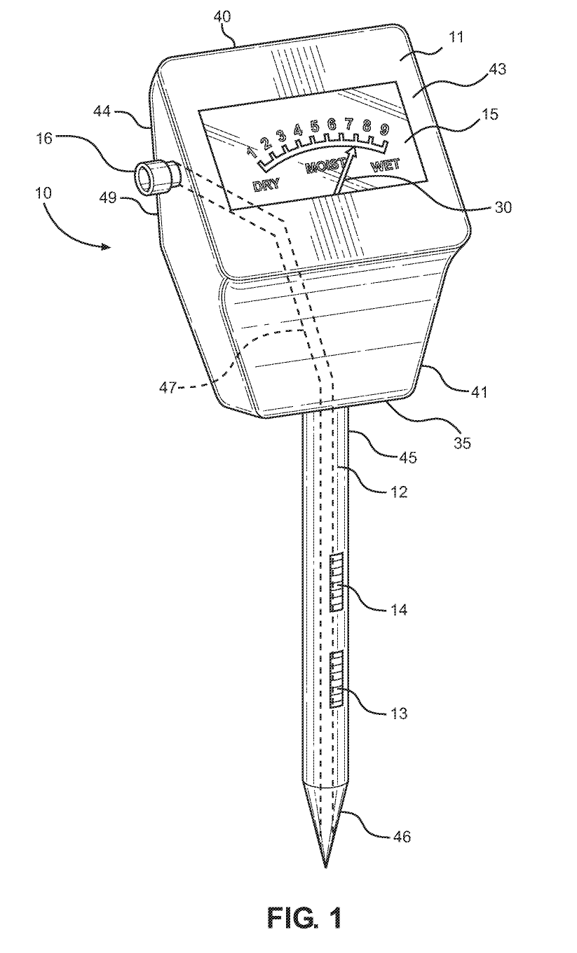

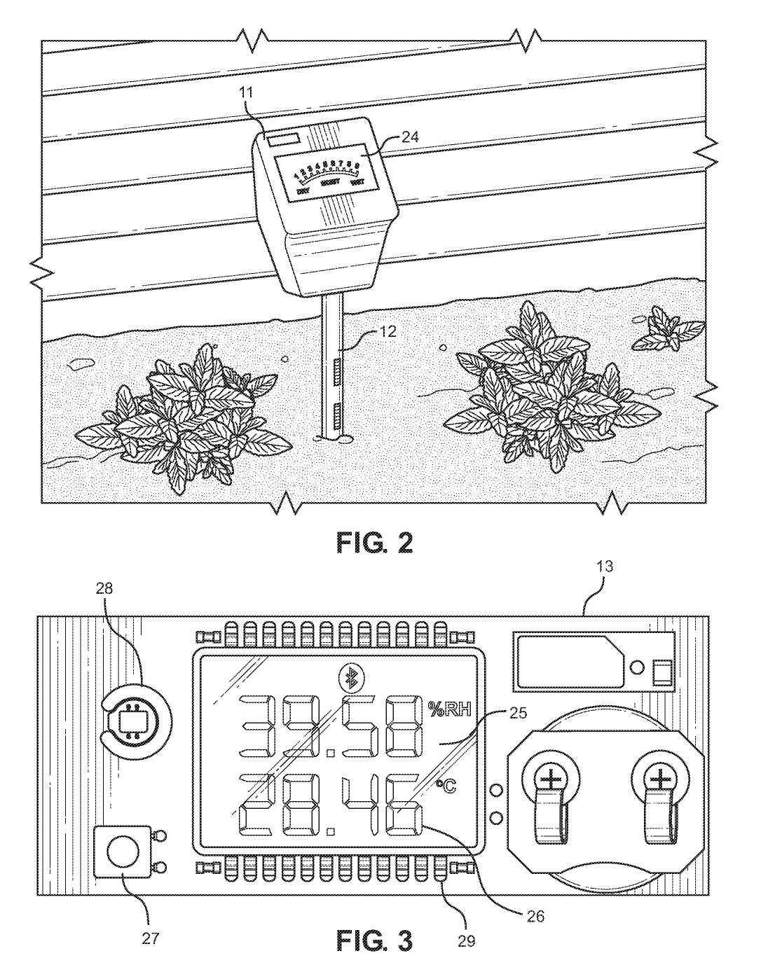

Referring now to FIG. 1 and FIG. 2 there is shown a perspective view of an embodiment of the moisture meter and a perspective view of an embodiment of the moisture meter in use. In the illustrated embodiment, the moisture meter 10 is comprised of a housing 11 having a stake 12 disposed at a lower end 35 thereof. The moisture meter 10 further comprises a moisture sensor 13 and temperature sensor 14 adjacent to each other and disposed on a lower, outside perimeter of the stake 12. A valve 16 is disposed on the housing 11 wherein a water source can be removably secured to the valve 16. The housing 11 is includes of a top portion 40 and a bottom portion 41 with a front portion 43 and rear portion 44. In the illustrated embodiment, the housing is composed of a hard, plastic material. The housing 11 further comprises a measuring display 15 disposed on the front portion 43 that is made of a glass, hard plastic or plexiglass material wherein a moisture scale is disposed thereon. In the shown embodiment, the housing 11 is a rectangular shape however the housing 11 can be any suitable shape such as a square or circle.

The stake comprises a top portion 45 and a bottom portion 46, wherein the bottom portion 46 forms a point and is adapted to engage the ground providing easier insertion therethrough. The height and width of the stake 12 can be any suitable measurement depending on the soil density. Bulk density is an indicator of soil compaction which reflects the soil's ability to function for water movement and structural support, among other things. As a result, the length of the stake 12 may require adjustment depending on the bulk density of the soil. The stake 12 further comprises a channel 47 disposed therein and extending between the valve 16 and the bottom portion 46 of the stake 12, allowing liquid to flow through an aperture along the bottom portion 46 and into the ground.

The moisture meter 10 further comprises the measuring display 15 wherein the measuring display 15 is in operable communication with the moisture sensor and communicates a level of soil moisture to a user via wireless transmitting capabilities. In some embodiments, the level is equivalent to a percentage of moisture detected by the moisture sensor. In other embodiments, the level is equivalent to pH detected by a pH sensor. In alternate embodiments, the display can show both the pH and percent moisture detected in the soil. In the shown embodiment, the level of pH and an indication of moisture level is simultaneously displayed. The display includes a pH range from 1 to 9 with 1 being acidic and 9 being basic. Further, beneath the pH reading, the indicia of moisture level is displayed. The measuring display 15 displays the moisture level through the words, “dry” “moist” and “wet”, respectively, disposed thereon corresponding to the levels of soil moisture and further establishing the level of soil moisture. In the illustrated embodiment, “dry” includes a range of 0-35% soil moisture detected by the moisture sensor, “moist” includes a range of 36-74% soil moisture detected by the moisture sensor, and “wet” includes a range of 75-100% soil moisture.

The measuring display 15 further comprises a dial 30 configured to move to an appropriate soil moisture number based on the level of soil moisture wherein the moisture sensor is configured to communicate the moisture level to the measuring display 15. In alternate embodiments, the level of soil moisture can be any suitable number range and any suitable measuring display 15, such as digital.

In the illustrated embodiment, when the pH level communicates a value of level 5, the valve is actuated such that water is allowed to flow through the channel and into the ground. The valve will also open when a temperature condition exceeds 90 degrees F. Data of air temperature and soil pH were collected over a 7-day period. Table 1 illustrates the data wherein the soil pH and air temperature were measured at 6-hour intervals throughout the 7 days. During the 7-day period, the valve opened a total of nine times—at results 4, 8, 11, 12, 15, 16, 20, 24, and 28. The pH and temperature was detected by sensors disposed on the stake. In alternate embodiments, the moisture percentage of the soil is also recorded.

TABLE 1

| Result | Date | Time | | pH Level | Temperature (° F.) |
|---|---|---|---|---|---|
| 1 | Day 1 | 12:00 | AM | 7.2 | 60 |
| 2 | Day 1 | 6:00 | AM | 6.1 | 65 |
| 3 | Day 1 | 12:00 | PM | 5.1 | 75 |
| 4 | Day 1 | 6:00 | PM | 4.2 | 72 |
| 5 | Day 2 | 12:00 | AM | 7.2 | 59 |
| 6 | Day 2 | 6:00 | AM | 6.5 | 62 |
| 7 | Day 2 | 12:00 | PM | 5.5 | 82 |
| 8 | Day 2 | 6:00 | PM | 4.6 | 75 |
| 9 | Day 3 | 12:00 | AM | 6.1 | 65 |
| 10 | Day 3 | 6:00 | AM | 5.5 | 65 |
| 11 | Day 3 | 12:00 | PM | 4 | 77 |
| 12 | Day 3 | 6:00 | PM | 3.5 | 87 |
| 13 | Day 4 | 12:00 | AM | 5.9 | 57 |
| 14 | Day 4 | 6:00 | AM | 5 | 68 |
| 15 | Day 4 | 12:00 | PM | 4.6 | 78 |
| 16 | Day 4 | 6:00 | PM | 4 | 88 |
| 17 | Day 5 | 12:00 | AM | 7.1 | 57 |
| 18 | Day 5 | 6:00 | AM | 6.6 | 62 |
| 19 | Day 5 | 12:00 | PM | 5.5 | 72 |
| 20 | Day 5 | 6:00 | PM | 4.5 | 82 |
| 21 | Day 6 | 12:00 | AM | 6.7 | 62 |
| 22 | Day 6 | 6:00 | AM | 5.9 | 70 |
| 23 | Day 6 | 12:00 | PM | 5.2 | 75 |
| 24 | Day 6 | 6:00 | PM | 4.7 | 84 |
| 25 | Day 7 | 12:00 | AM | 5.9 | 75 |
| 26 | Day 7 | 6:00 | AM | 5.4 | 62 |
| 27 | Day 7 | 12:00 | PM | 4.9 | 77 |
| 28 | Day 7 | 6:00 | PM | 4 | 82 |

In the shown embodiment, the valve 16 is disposed on a side 49 portion of the housing 11 and is adapted to receive a water dispenser therein. The valve is made of a hard, plastic material and the water dispenser is any suitable water dispenser, such as a hose or a sprinkler. In some embodiments, the valve 16 is operably connected to the moisture and temperature sensors 13, 14, such that the valve 16 is configured to automatically actuate, allowing water to flow through the stake 12 when the moisture and temperature readings reach a pre-set level. The pre-set level is dependent on the soil type and the temperature reading. In the illustrated embodiment the valve resembles a sprinkler valve is that the valve is actuated by an electrical signal received by the moister sensor when a pre-set level is detected.

Referring now to FIG. 3, there is shown a close-up view of the temperature sensor and moisture sensor of the moisture meter. In the shown embodiment, the moisture sensor 13 is configured to detect moisture information regarding the amount of moisture present in soil and configured to transmit the moisture level information to the measuring display 15, wherein the measuring display 15 transmits the moisture information wirelessly via a wireless transmitter 17 located in the housing 11.

The moisture sensor 13 further comprises at least one insulated electrode 28 structured for insertion into the soil to a predetermined depth and a measurement circuitry 29 operably connected to the insulated electrode 28 and configured to provide a measurement of the moisture level in the soil. In the illustrated embodiment, the predetermined depth is approximately 6 inches into the soil measured from the surface of the soil. The moisture level is also digitally displayed on an outside perimeter 25 of the moisture sensor 13.

In some embodiments, the shown display will also be identically displayed on a web application. In the shown embodiment, the moisture sensor 13 comprises a power source 27, such as a battery or a solar cell to make the moisture sensor 13 operable; however, any suitable power source may be used.

In addition, the temperature sensor 14 is operable to detect a temperature of the air and configured to provide an electrical signal dependent on the temperature of the soil to the measuring display 15, wherein the measuring display 15 transmits the temperature information wirelessly via the wireless transmitter 17 in the housing 11. The temperature is also digitally displayed on an outside perimeter 26 of the temperature sensor 14.

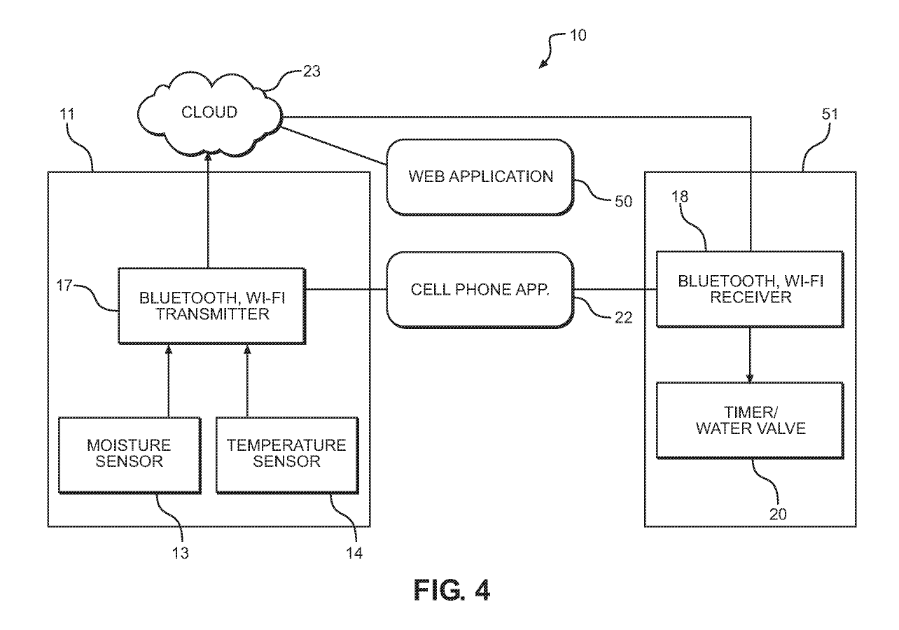

Referring now to FIG. 4, there is shown a schematic diagram of the moisture meter. The wireless transmitter 17 disposed within the housing 11 is configured to transmit the moisture, pH, and temperature readings from the moisture, pH, and temperature sensors 13, 14 to an external device using IoT. In some embodiments, the external device comprises a mobile phone, while in another embodiment, the external device comprises a web application 50 or database. The wireless transmitter 17 is further configured to wirelessly send notifications to an external device should the moisture or temperature readings reach a pre-set level. The housing 11 further comprises a wireless transmitter 17 disposed therein, wherein the wireless transmitter is configured to transmit the moisture and temperature readings to a cell phone application 22 and an external cloud source 23. Wireless receiver 18 is configured to receive valve actuation instruction from an external source such as web application 50 and send it to timer on an outside water valve 20, wherein the water valve 20 is adapted to actuate water flow when a pre-set time is reached.

In the illustrated embodiment, the wireless receiver 18, the timer and water valve 20 are disposed within a single housing 51. The pre-set time can be any suitable time selected by the user. The external web application is programmable by user, wherein the user selects the pre-set moisture levels and temperature readings for actuation of the valve. In operation, the moisture and temperature sensors transmit via the wireless transmitter information detected thereby to the external web application, if the web application determines that the moisture level and temperature reading are at the pre-set output, a signal is sent to the wireless receiver disposed in the valve housing to actuate the valve for a predetermined period of time via the timer.

Moisture and temperature data detected from the sensors are sent via the transmitter 17 to a server a predetermined number of times per day. In the illustrated embodiment, the data is collected and transmitted 4 times per day at 6-hour intervals, as shown in Table 1. Depending if the measurements exceed the threshold value of predetermined levels, the server will send a signal to the water valve 20 on TCP/IP connection in order to automatically open the valve and release water therethrough. The duration of the valve remaining open and at what times the measurements are transmitted to the server are set on a user interface. Alternatively, the timing can be manually set on a timer disposed on the water valve controls. Further, the user is able to select the predetermined levels upon which the valve will open through the web application. For example, if a user desires to conserve water, the user will select a lower pH level i.e. 4 or moisture level i.e. “dry.” In the illustrated embodiment, the housing and sensors does not need to be calibrated because a user is able to manually calibrate through the web interface.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A moisture meter, comprising:
a housing having a stake disposed at a lower end thereof;
a moisture sensor disposed on a perimeter of the stake configured to detect moisture in soil;
a temperature sensor disposed on the perimeter of the stake configured to detect a temperature of the soil;
a valve operably connected to a water source, wherein the water source is removably secured to the valve;
a wireless transmitter disposed within the housing configured to send temperature and moisture information detected by the moisture sensor and the temperature sensor to an external source;
a wireless receiver configured to actuate the valve allowing water to flow through the stake when the moisture and temperature sensors each detect a pre-set level.

2) The moisture meter of claim 1, wherein a channel is disposed between the valve and a tip of the stake, allowing liquid to flow therethrough.

3) The moisture meter of claim 1, wherein the external device is a cell phone application and a web application.

4) The moisture meter of claim 1, wherein the moisture meter is configured to be programmed to send a notification through the transmitter to an external source when the moisture and temperature sensors each detect a pre-set level.

5) The moisture meter of claim 1, wherein the valve is operably connected to the temperature and moisture sensors, such that the valve is configured to automatically actuate, allowing water to flow through the stake, when the moisture and temperature readings reach a pre-set level.

6) The moisture meter of claim 1, wherein the wireless receiver is disposed on the valve and configured to directly receive moisture and temperature readings from the wireless transmitter.

7) The moisture meter of claim 1, further comprising at least one insulated electrode configured to insert into the soil to a predetermined depth.

8) The moisture meter of claim 1, further comprising a measuring display configured to receive moisture information from the moisture sensor and transmit the moisture information wirelessly via the wireless transmitter.

9) The moisture meter of claim 8, the measuring display comprising a range of digits configured to indicate moisture level detected from the moisture sensor and a plurality of descriptive words to indicate corresponding moisture level detected by the moisture sensor.
